(19) United States
(12) Patent Application Publication
(10) Pub. No.: US 2017/0304281 A1
(43) Pub. Date: Oct. 26, 2017

(54) METHODS OF USING POTASSIUM CHANNEL INHIBITORS (BLOCKERS) FOR FLUID RESUSCITATION

(71) Applicant: Loyola University of Chicago, Maywood, IL (US)

(72) Inventors: Matthias Majetschak, Woodridge, IL (US); Kenneth Lee Byron, Chicago, IL (US); Sean Patrick Nassoiy, River Forest, IL (US)

(21) Appl. No.: 15/491,094

(22) Filed: Apr. 19, 2017

Related U.S. Application Data
(60) Provisional application No. 62/325,568, filed on Apr. 21, 2016.

(51) Int. Cl. A61K 31/444; A61K 9/00

(52) U.S. Cl. CPC A61K 31/444; A61K 9/0026

(57) ABSTRACT

Methods and pharmaceutical treatments of fluid resuscitation in which a selective potassium channel inhibitor is administered to a patient in a therapeutic amount sufficient to stabilize blood pressure and/or to reduce the amount of the resuscitation fluid otherwise required to resuscitate the patient.

### CROSS REFERENCE TO RELATED APPLICATIONS

This application claims the benefit of U.S. Provisional Application No. 62/325,568, filed Apr. 21, 2016, the contents of which are incorporated herein by reference.

### STATEMENT REGARDING FEDERALLY SPONSORED RESEARCH

This invention was made with government support under Contract Nos. R01GM107495-01A and T32GM008750 awarded by the National Institutes of Health (National Institute of General Medical Sciences) and Contract No. W81XWH-15-1-0262 awarded by the Department of Defense. The government has certain rights in the invention.

### BACKGROUND OF THE INVENTION

The present invention generally relates to therapeutic strategies utilizing proteinacious channels in lipid membranes of mammalian cells. More particularly, this invention relates to the utilization of the electrophysiology of potassium channels and inhibitors (blockers) thereof as pharmaceutical treatments for fluid resuscitation, including but not limited to reducing resuscitation fluid requirements in hypotensive and/or hemodynamically unstable patients, for example, patients who have suffered severe hemorrhages.

Mammalian cells, including the smooth muscle cells within the walls of arteries (vascular smooth muscle cells, or VSMCs) are surrounded by a lipid membrane which functions as a barrier to diffusion of many soluble substances, including ions, into and out of the cytosol of the cells. Proteinacious channels integrated into these lipid membranes allow ions to cross the lipid membrane when the channels are open. A portion of these proteinacious channels is selective for potassium ions (K⁺), and are referred to as potassium channels or K⁺ channels. Still other proteinacious channels are selective for calcium ions (Ca²⁺), and are referred to as calcium channels or Ca²+ channels. Under normal circumstances, potassium ions (K⁺) are typically present inside the cell at concentrations of about twenty-five times higher as compared to their corresponding concentration outside the cell. When potassium channels open (activate), potassium ions (K⁺) tend to leak out of the cell through the potassium channels, resulting in a measurable electrical current across the membrane. This electrical current establishes an electrical charge difference across the lipid membrane (membrane voltage, or Vₘ), resulting in the polarization of the membrane.

VSMCs are able to contract or relax to regulate blood flow (and blood pressure). As noted above, K⁺ channels represent a primary effector for adjusting Vₘ in VSMCs. K⁺channels of the Kv7 family (also known as KCNQ) have been identified among the cohort of vascular ion channels. These Kv7 channels, which were previously recognized as mediators of acetylcholine-induced neuronal excitation, have distinctive electrophysiological characteristics: activation at voltages negative to −50 mV, outward rectification, and absence of time-dependent inactivation.

Kv7 voltage-activated potassium channels contribute to the regulation of the membrane potential in excitable cells. See, for example, Delmas et al., “Pathways modulating neural KCNQ/M (Kv7) potassium channels,” Nat Rev Neurosci 6(11):850-862 (2005); and Robbins et al., KCNQ potassium channels: physiology, pathophysiology, and pharmacology,” Pharmacol Ther 90(1):1-19 (2001). KCNQ5 (Kv7.5) channels have been determined to be expressed and functional in vascular smooth muscle cells. U.S. Pat. Nos. 8,785,466, 8,686,017, and 9,326,955 represent examples of discoveries relating to the use of Kv7 channels to identify pharmaceutical treatments and assess risks for a variety of medical conditions. Recent evidence suggests that pharmacological inhibition of Kv7 channel activity may enhance blood vessel contractility in vitro. Furthermore, Kv7 channel inhibitors (blockers) have been described to reduce cardiac ischemia-reperfusion injury in an isolated perfused rat heart model (Hedegaard et al., J Pharmacal Exp Ther 2016).

Adequate fluid resuscitation to compensate for intravascular volume deficits and to support organ perfusion is an essential cornerstone in the treatment of critically ill patients, including patients undergoing major surgery and patients with severe burns, trauma, hemorrhage, or sepsis. Current resuscitation strategies have been limited to fluid resuscitation (crystalloid, colloid), vasopressor treatment, and blood transfusion/surgical intervention if hemorrhage is a contributing factor. Fluid resuscitation carries the well-recognized risk of fluid overload, which can lead to third-spacing of fluids into tissues, edema formation (lung, bowel), coagulopathy, compartment syndrome, or acute lung injury, and significantly contributes to mortality and morbidity in critically ill patients. In patients undergoing major surgery or in sepsis patients who fail to meet resuscitation targets despite aggressive fluid resuscitation, vasopressors (as examples, catecholamines and arginine vasopressin) are commonly added. These drugs, however, can have significant adverse effects and their use is limited by vasoconstrictor-induced ischemia. Furthermore, vasopressor refractoriness develops frequently with prolonged use of vasoactive drugs, leading to increased dosing requirements and significantly increased risks for adverse effects, such as bowel ischemia. Novel therapeutics capable of reducing fluid resuscitation requirements and that avoid vasopressor-induced morbidity and mortality would be highly desirable.

The effects of drugs that modulate Kv7 channel activity on fluid resuscitation in vivo are unknown. WO2008134740A1 reports

In an embodiment, the substance includes at least one of the agents selected from the group consisting of an agent that alters potassium levels, an agent that alters calcium levels, an agent that reduces activation of cardiac beta receptors, and an agent that reduces mitochondrial electron transport.

an embodiment of the present disclosure, distinct substances or agents or combinations of agents are administered to a subject suffering from ischemia or an ischemic event. Such agents may include agents involved in altering potassium levels and/or calcium levels such as potassium and calcium channel blockers, agents involved in reducing activation of cardiac beta receptors such as beta blockers, and agents involved in reducing mitochondrial electron transport. In addition, myosin inhibitors such as 2,3-butanedione monoxime may be that block ATP and calcium binding to actin-myosin may be administered to the subject.

WO2008134740A1 discloses the prevention from ischemic injury in “a heart, a brain, liver, pancreas, kidney or gastro-intestinal organ.” The use of Kv7 channel modulators as a component of fluid resuscitation strategies in non-ischemic conditions, such as hypovolemic shock, as described hereinafter has not been described previously.

In view of the above, it can be appreciated that there is an ongoing desire for improved fluid resuscitation processes, and that it would be desirable if drugs were available that reduced the fluid resuscitation requirements of patients without the side effects of current drugs, such as vasopressors.

### BRIEF DESCRIPTION OF THE INVENTION

The present invention provides methods of using potassium channel inhibitors (blockers) as pharmaceutical treatments for fluid resuscitation, including but not limited to reducing resuscitation fluid requirements in hypotensive and/or hemodynamically unstable patients.

According to one aspect of the invention, a method of fluid resuscitation is provided that uses a pharmaceutical treatment in which a selective potassium channel inhibitor is administered to a patient in a therapeutic amount sufficient to stabilize blood pressure of the patient.

According to another aspect of the invention, a pharmaceutical treatment is provided for patients who are hypotensive and/or hemodynamically unstable and undergoing fluid resuscitation with a resuscitation fluid. The pharmaceutical treatment involves administering a selective Kv7 potassium channel inhibitor to the patient in a therapeutic amount sufficient to reduce the amount of the resuscitation fluid otherwise required to resuscitate the patient.

A technical effect of the invention is the ability to reduce systemic fluid requirements to stabilize cardiovascular function and maintain hemodynamics during resuscitation of critically ill patients, and in doing so preferably and significantly reduce morbidity and mortality from fluid-overload.

Other aspects and advantages of this invention will be better appreciated from the following detailed description.

### BRIEF DESCRIPTION OF THE DRAWINGS

FIG. 1 is a plot representing effects of the selective Kv7 channel inhibitor linopirdine on blood pressure in normal animals.

FIG. 2 is a plot representing effects of the Kv7 channel activator retigabine on blood pressure in normal rats.

FIG. 3 contains three plots indicating that linopirdine reduced resuscitation fluid requirements after hemorrhagic shock in rats.

FIG. 4 contains three plots indicating that XE991, a structural analogue of linopirdine with higher potency for Kv7 channel blockade, reduced resuscitation fluid requirements after hemorrhagic shock in rats.

FIG. 5 contains three plots indicating that supplementation of resuscitation fluids with linopirdine reduced resuscitation fluid requirements after hemorrhagic shock in rats at a ten-fold reduced cumulative dose, when compared with bolus administration.

### DETAILED DESCRIPTION OF THE INVENTION

As discussed below, the present invention arises in part from an investigation indicating that administration of a selective Kv7 potassium channel inhibitor (blocker), for example, linopirdine or XE991, is able to reduce resuscitation fluid requirements in hypotensive and/or hemodynamically unstable subjects (e.g., rats), and induces small and transient increases in blood pressure only if administered in sufficiently high doses. The structure, composition, and manufacture of Kv7 potassium channel inhibitors (blockers) are well known in the art and therefore will not be explained further herein.

Initial phases of the investigation entailed the administration of linopirdine to anesthetized male Sprague-Dawley rats to determine its effect on blood pressure. Hemodynamics and fluid requirements were continuously monitored.

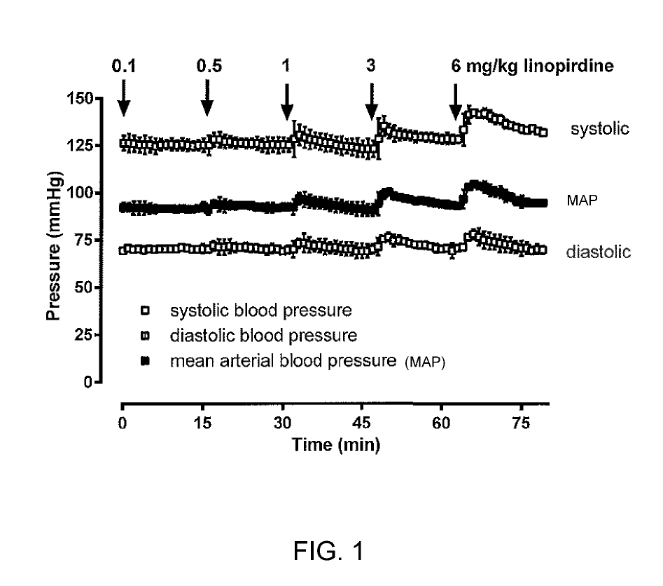

FIG. 1 contains a plot representing the effect of linopirdine on blood pressure in normal rats (sham—no hemorrhage, n=3) that received five increasing intravenous (i.v.) bolus doses of linopirdine (0.1-6 mg per kg of body weight) in 0.5 mL normal saline (NS) at fifteen minute intervals. FIG. 1 indicates that linopirdine induced small and transient increases in blood pressure only when administered in high dosages. In particular, from a baseline mean arterial blood pressure (MAP) of 92±2.5 mmHg, MAP peaked at 92±2 mmHg, 93±2 mmHg, 95±4 mmHg, 100±2 mmHg and 105±0.6 mmHg with linopirdine dosages of 0.1 mg/kg, 0.5 mg/kg, 1 mg/kg, 3 mg/kg and 6 mg/kg, respectively, and returned to baseline within about ten to fifteen minutes. At the 6 mg/kg dosage, linopirdine injection transiently increased mean arterial blood pressure by approximately 10%. Data are mean±SD.

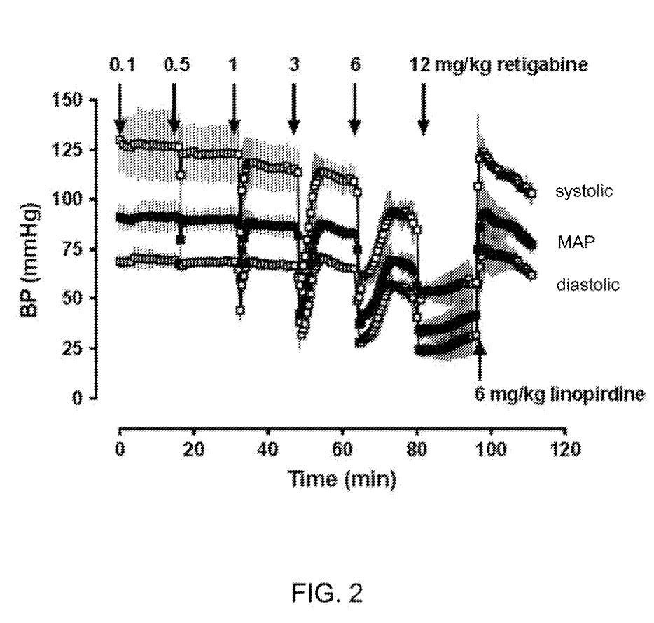

FIG. 2 contains a plot representing the effect of the Kv7 channel activator retigabine on blood pressure when administered to normal rats (sham—no hemorrhage, n=3) by intravenous injection in increasing dosages of 0.1 mg/kg, 0.5 mg/kg, 1 mg/kg, 3 mg/kg, 6 mg/kg, and 12 mg/kg in 0.5 mL normal saline. FIG. 2 indicates that retigabine dose-dependently reduced blood pressure. Hypotension resulting from the last dosage of retigabine was able to be reversed with an injection of 6 mg/kg linopirdine. Data are mean±SD.

Further phases of the investigation entailed the use of anesthetized male Sprague-Dawley rats that were hemorrhaged to a mean arterial blood pressure (MAP) of 25 mmHg for thirty minutes to induce hemorrhagic shock, followed by fluid resuscitation with normal saline to a MAP of 70 mmHg until t=75 minutes. Hemodynamics and fluid requirements were continuously monitored.

Based on these series of investigations, it was concluded that even a high intravenous bolus dose of linopirdine (6 mg/kg) and a total intravenous dose of 10.6 mg/kg of linopirdine administered within one hour caused only minimal and short-lived increases in systemic blood pressures, suggesting that systemic vasopressor effects of linopirdine are consistently small. Despite only modest effects of acute linopirdine treatment on blood pressure in normotensive rats, it was observed that retigabine-induced hypotension was instantaneously reverted with intravenous linopirdine. As such, it was concluded that Kv7 channel modulators may provide an alternative pharmacological approach for the management of hypertensive emergencies, in which drugs that permit rapid, titratable and reversible reduction of blood pressure are highly desirable.

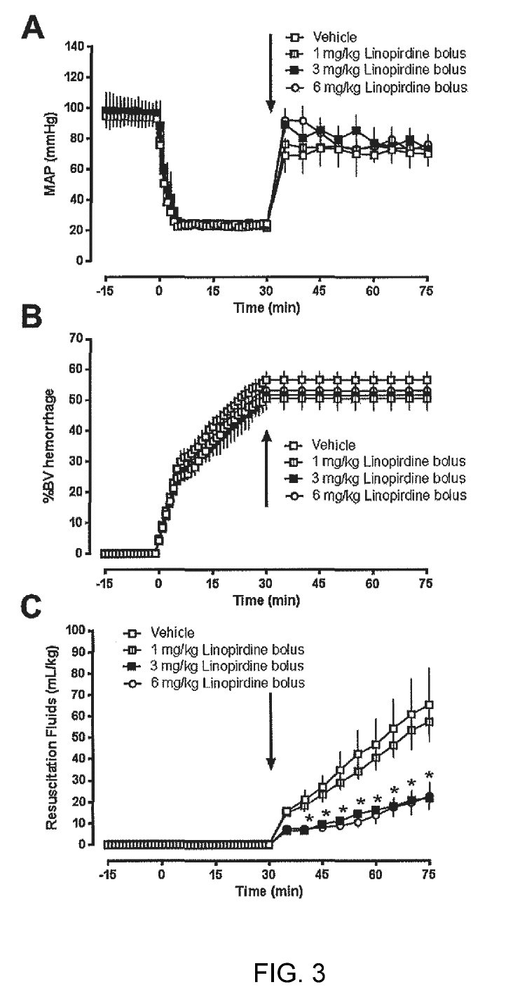

FIG. 3 contains three plots evidencing the ability of linopirdine to dose-dependently reduce fluid requirements to maintain hemodynamics during resuscitation from induced hemorrhagic shock. At the end of the hemorrhagic shock period (t=30 min), normal saline alone (“vehicle”; n=4), 1 mg/kg (n=5), 3 mg/kg (n=3), or 6 mg/kg (n=3) linopirdine in 0.5 mL normal saline were bolus injected intravenously. Plot “A” represents the mean arterial blood pressure (MAP, mmHg), plot “B” represents the hemorrhage volume in percent of total blood volume, and plot “C” represents the resuscitation fluid requirements to maintain blood pressure at 70 mmHg. Fluid requirements to resuscitate to a MAP of 70 mmHg were: 65±34 mL/kg with normal saline alone and 57±13 mL/kg, 22±8 mL/kg (p<0.05 vs. normal saline alone) and 22±11 mL/kg (p<0.05 vs. normal saline alone) with 1 mg/kg, 3 mg/kg, and 6 mg/kg linopirdine, respectively. The effect of linopirdine to reduce fluid requirements to maintain hemodynamics during resuscitation from hemorrhagic shock appeared to be saturated at a bolus dose of about 3 mg/kg linopirdine. As compared with normal saline-treated animals, a bolus injection of linopirdine (3 mg/kg and 6 mg/kg) reduced fluid resuscitation requirements by 70%. Data are mean±SD. *:p<0.05 vs. normal saline alone. Two-way ANOVA/Bonferroni post-hoc multiple comparison test.

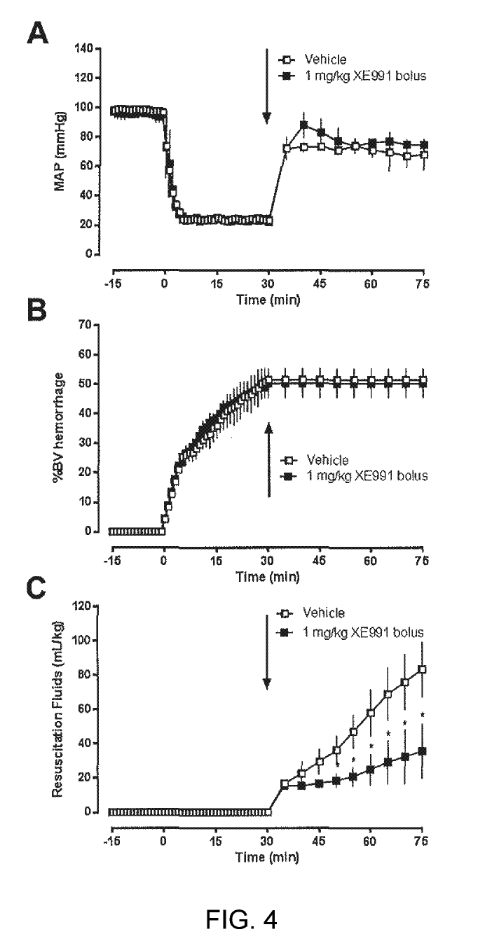

FIG. 4 contains three plots evidencing the ability of another Kv7 channel inhibitor, XE991 (a structural analogue of linopirdine with five to ten-fold higher potency for channel blockade) to reduce fluid requirements to maintain hemodynamics during resuscitation from induced hemorrhagic shock. At the end of the hemorrhagic shock period (t=30 min), normal saline alone (“vehicle”; n=3) or 1 mg/kg (n=3) of XE991 in 0.5 mL normal saline were injected intravenously. Plot “A” represents the mean arterial blood pressure (MAP, mmHg), plot “B” represents the hemorrhage volume in percent of total blood volume, and plot “C” represents the resuscitation fluid requirements to maintain blood pressure at 70 mmHg. In agreement with its higher potency to block Kv7 channels, plot C evidences that XE991 at a bolus dose of 1 mg/kg had a comparable effect to linopirdine at bolus doses of 3 mg/kg and 6 mg/kg (see Plot C of FIG. 3). Data are mean±SD. *:p<0.05 vs. normal saline alone. Two-way ANOVA/Bonferroni post-hoc multiple comparison test.

The investigations discussed above indicated that a single dose of linopirdine at the beginning of fluid resuscitation from hemorrhagic shock dose-dependently reduced fluid requirements to stabilize blood pressure. The observed effects of linopirdine were saturated at a dose of three milligrams per kilogram and resulted in 65% reduction of resuscitation fluid requirements. The observation that a dose of one milligram per kilogram of the linopirdine analogue XE991 was equally efficacious to reduce fluid resuscitation requirements as a dose of three milligrams per kilogram of linopirdine is consistent with the higher in vitro and in vivo potency of XE991. It was concluded that these data suggested that the fluid-sparing effects of linopirdine and XE991 during resuscitation from hemorrhagic shock can be considered as a general pharmacological property of drugs that block Kv7 currents.

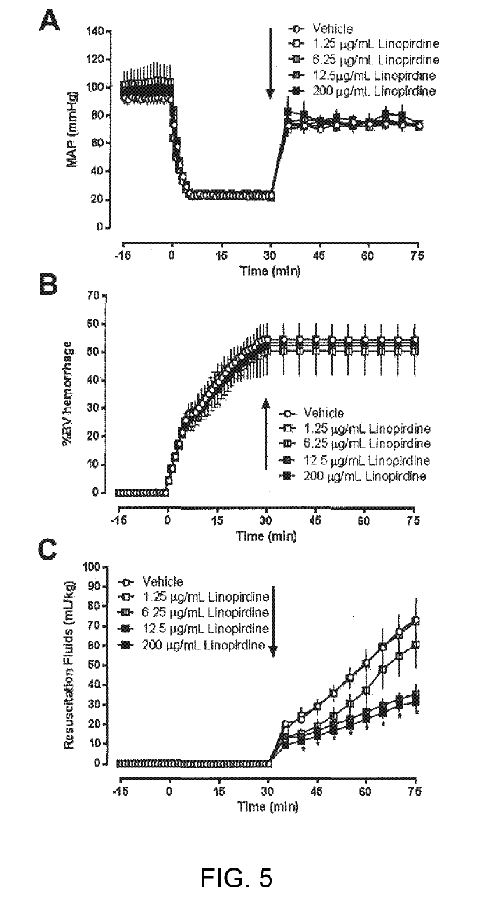

FIG. 5 contains three plots evidencing that supplementation of a resuscitation fluid (normal saline) with linopirdine dose-dependently reduced resuscitation fluid requirements after induced hemorrhagic shock in rats at a ten-fold reduced cumulative dose, when compared with bolus administration. At the end of the hemorrhagic shock period (t=30 min), animals were resuscitated with normal saline alone (“vehicle”; n=4) or normal saline supplemented with 1.25 (n=4), 6.25 (n=3), 12.5 (n=4), or 200 (n=6) pg/mL of linopirdine. Plot “A” represents the mean arterial blood pressure (MAP, mmHg), plot “B” represents the hemorrhage volume in percent of total blood volume, and plot “C” represents the resuscitation fluid requirements to maintain blood pressure at 70 mmHg. Fluid requirements to resuscitate to a MAP of 70 mmHg were: 73±12 mL/kg with NS, 72±24 mL/kg with NS supplemented with 1.25 μg/mL linopirdine and 61±20 mL/kg, 36±9 mL/kg (p<0.05 vs. NS alone) and 31±9 mL/kg (p<0.05 vs. NS alone) with NS supplemented with 6.25 μg/mL, 12.5 μg/mL and 200 μg/mL linopirdine, respectively. The effect of linopirdine was saturated when the normal saline was supplemented with 12.5 μg/mL linopirdine and comparable to a bolus injection of 3 mg/kg and 6 mg/kg linopirdine. Significantly, with normal saline supplemented with 12.5 μg/mL linopirdine, the cumulative dose of linopirdine was 0.446 mg/kg, which was equi-efficacious as a single bolus injection of 3 mg/kg and 6 mg/kg (see Plot C of FIG. 3). Data are mean±SD. *:p<0.05 vs. normal saline alone. Two-way ANOVA/Bonferroni post-hoc multiple comparison test.

No toxicity associated with linopirdine treatment was observed in the investigations leading to the present invention. It has previously been determined that after oral administration in humans, the half-life of linopirdine is about 0.4 to 3.2 hours. After intravenous injection of 2.5 mg/kg linopirdine, a half-life of 0.6 hours has been determined in rats. Thus, the short half-life would indicate that linopirdine is a drug that is easily controllable if adverse effects were to occur.

On the basis of the above investigations, it was concluded that selective Kv7 potassium channel inhibitors (blockers) can be administered in a dose-dependent manner to stabilize blood pressure and reduce systemic fluid requirements to maintain hemodynamics during resuscitation after hemorrhagic shock in a subject. The data support the use of potassium channel inhibitors as a new pharmacological approach to improve fluid resuscitation strategies after severe hemorrhagic shock. Such pharmacological approaches would have the capability of significantly reducing morbidity and mortalityfrom fluid-overload and reducing the need for vasopressor support. Although not yet tested on humans, the expression pattern of Kv7 channels is very similar across species, with Kv7.1, Kv7.4, and Kv7.5 being ubiquitously expressed in every arterial bed so far examined (see, Haick et al., Novel treatment strategies for smooth muscle disorders: Targeting Kv7 potassium channels. Pharmacol Ther. 2016;165:14-25). Therefore, the above investigations indicate that Kv7 potassium channel inhibitors (blockers) can be administered to stabilize blood pressure and reduce systemic fluid requirements during resuscitation treatments in humans.

More generally, the investigation reported above indicated that Kv7 channel inhibitors may find use as a supplement for various resuscitation fluids such as Lactated Ringer's solution or other resuscitation fluids for out-of hospital and in-hospital resuscitation, for trauma, burn, sepsis, or shock resuscitation, for resuscitation during major cardiovascular, abdominal or transplant surgery, and for resuscitation of patients with medical conditions associated with limited fluid tolerance, including but not limited to kidney failure or heart diseases (myocardial insufficiency, myocardial infarction, congestive heart failure, cardiomyopathy, etc.).

The dose of the pharmaceutical administered to a subject, particularly a human, in the context of the present invention, should be sufficient to effect a therapeutic response in the subject over a reasonable time frame. One skilled in the art will recognize that dosage will depend upon a variety of factors including a condition of the subject, the body weight of the subject, the nature and extent of the symptoms, the kind of concurrent treatment, the frequency of treatment, etc. The size of the dose also will be determined by the route, timing and frequency of administration as well as the existence, nature, and extent of any adverse side effects that might accompany the administration of the pharmaceutical and the desired physiological effect. Appropriate dosing may be determined empirically from clinical trials, starting with doses that have established safety profiles when used for other applications. For example, Linopirdine has previously been tested in phase 1 and phase 2 clinical trials as a cognition-enhancing drug in Alzheimer's disease with no relevant adverse effects being reported, and linopirdine dosages evaluated in the investigation reported herein were comparable with or below those shown to be safe in humans in those clinical trials. Such an advantageous pharmacological profile would permit rapid transition of linopirdine into clinical trials. It is foreseeable and within the scope of the invention that the dose may be administered via intravenous bolus injection or as supplementation of resuscitation fluids as shown herein, or by another method known in the art.

While the invention has been described in terms of a particular investigation, it is apparent that other forms could be adopted by one skilled in the art. For example, it is foreseeable that uses could be determined for other potassium channel inhibitors (blockers) as pharmaceutical treatments for fluid resuscitation of patients suffering from a variety of conditions. Furthermore, potassium channel inhibitors could be administered with various resuscitation fluids used in patients, for example, crystalloid solutions (including normal, isotonic, and hypotonic saline solutions), colloid solutions, blood, blood products, blood substitutes, etc. Accordingly, it should be understood that the invention is not limited to any embodiment described herein. It should also be understood that the phraseology and terminology employed above are for the purpose of describing the disclosed investigations, and do not necessarily serve as limitations to the scope of the invention. Therefore, the scope of the invention is to be limited only by the following claims.

1. A method of fluid resuscitation using a pharmaceutical treatment comprising administering a selective potassium channel inhibitor to a patient in a therapeutic amount sufficient to stabilize blood pressure of the patient.

2. The method of claim 1, wherein the patient is hypotensive and/or hemodynamically unstable, the patient is undergoing fluid resuscitation, and the administering of the selective potassium channel inhibitor reduces fluid resuscitation requirements for the patient.

3. The method of claim 1, wherein the patient is in hemorrhagic shock when the selective potassium channel inhibitor is administered to the patient.

4. The method of claim 1, wherein the selective potassium channel inhibitor is administered to the patient while the patient is suffering from trauma, burn, or sepsis, or is undergoing cardiovascular, abdominal, or transplant surgery, or has a medical condition associated with limited fluid tolerance.

5. The method of claim 1, wherein the selective potassium channel inhibitor is administered via intravenous bolus injection.

6. The method of claim 1, wherein the selective potassium channel inhibitor is administered via supplementation of a resuscitation fluid.

7. The method of claim 6, wherein the resuscitation fluid is a crystalloid solution, a colloid solution, blood, a blood product, or a blood substitute.

8. The method of claim 1, wherein the selective potassium channel inhibitor is a Kv7 potassium channel inhibitor.

9. The method of claim 8, wherein the Kv7 potassium channel inhibitor is linopirdine.

10. The method of claim 8, wherein the Kv7 potassium channel inhibitor is XE991.

11. A pharmaceutical treatment of a patient who is hypotensive and/or hemodynamically unstable and undergoing fluid resuscitation with a resuscitation fluid, the pharmaceutical treatment comprising administering a selective Kv7 potassium channel inhibitor to the patient in a therapeutic amount sufficient to reduce the amount of the resuscitation fluid otherwise required to resuscitate the patient.

12. The pharmaceutical treatment of claim 11, wherein the selective Kv7 potassium channel inhibitor is administered to the patient while the patient is suffering from trauma, burn, or sepsis, or is undergoing cardiovascular, abdominal, or transplant surgery, or has a medical condition associated with limited fluid tolerance.

13. The pharmaceutical treatment of claim 11, wherein the selective Kv7 potassium channel inhibitor is administered via intravenous bolus injection.

14. The pharmaceutical treatment of claim 11, wherein the selective Kv7 potassium channel inhibitor is administered via supplementation of a resuscitation fluid.

15. The pharmaceutical treatment of claim 11, wherein all drugs administered during the pharmaceutical treatment lack significant intrinsic vasopressor activity.
